(19) United States
(12) Patent Application Publication
(10) Pub. No.: US 2025/0228695 A1
(43) Pub. Date: Jul. 17, 2025

(54) OSTOMY BARRIER APPLIANCE WITH SELF-ACTIVATING ADJUSTABLE CONVEXITY

(71) Applicant: HOLLISTER INCORPORATED, Libertyville, IL (US)

(72) Inventor: Gregory J. Czaplewski, Glendale Heights, IL (US)

(73) Assignee: HOLLISTER INCORPORATED, Libertyville, IL (US)

(21) Appl. No.: 18/849,214

(22) PCT Filed: Apr. 25, 2023
(86) PCT No.: PCT/US23/66150

§ 371 Date: Sep. 20, 2024

Related U.S. Application Data
(60) Provisional application No. 63/363,516, filed on Apr. 25, 2022.

(51) Int. Cl. A61F 5/445; A61F 5/448

(52) U.S. Cl. CPC A61F 5/445; A61F 2005/4483

(57) ABSTRACT

A convex ostomy barrier assembly for attaching an ostomy appliance to a peristomal skin surrounding a stoma includes a skin barrier, an inlet opening defined in the skin barrier for receiving the stoma, and a convexity adjusting device. The convexity adjusting device is configured to provide a convexity to the skin barrier and to automatically adjust the convexity according to the topography of user's peristomal skin as the convex ostomy barrier assembly is applied to a user. The convexity adjusting device is configured to provide a localized and customized adjustment of at least one characteristic of the convexity.

### BACKGROUND

This disclosure is related to a convex ostomy barrier. More particularly, the present disclosure pertains to an ostomy barrier appliance having an adjustable convexity feature.

Ostomy pouches for collecting bodily waste are used by individuals who have had surgery such as a colostomy, ileostomy, or urostomy. An ostomy pouch may be attached to a user via an ostomy barrier, which is configured to seal against peristomal skin surfaces and protect the peristomal surfaces from exposure to stomal effluent. However, the topography of stomas and peristomal surfaces surrounding stomas varies among patients, and sealing an ostomy appliance against such different peristomal surfaces and stomas remain as an area for further improvements. For example, a stoma may protrude more or less, or may even be flush or recessed.

A person with an ostomy having a stoma that is flush or recessed may find that applying external support or pressure from a barrier in the peristomal region aids in directing the discharge of effluent from the stoma directly into the ostomy pouch. Accordingly, the effectiveness of an adhesive seal between the ostomy barrier and the peristomal skin surface (i.e., a seal formed by the adhesive layer) may be prolonged. Thus, convex inserts and convex ostomy barriers, such as ADAPT® convex barrier rings available through the Applicant of the present application, have been developed to apply pressure around such peristomal regions.

However, the convexity of a conventional convex ostomy barrier or insert is fixed and may not work efficiently for all ostomates. Thus, convex ostomy barriers and convex inserts of various convexity depths have been made available in the market. Further, US 2019/0231580, which is assigned to the Applicant of the present application and incorporated herein by reference, discloses ostomy barrier appliances including a convexity adjusting device, which is configured to allow a user to adjust the convexity depth of a skin barrier. However, these ostomy barrier systems are configured to adjust only the convexity depth uniformly about the skin barrier and rely on user's intervention to correctly select or adjust the convexity depth of the ostomy barrier.

Accordingly, it is desirable to provide an improved ostomy barrier appliance that requires minimal user intervention to adjust various characteristics of the barrier convexity according to the topography of user's stoma and peristomal surface.

### BRIEF SUMMARY

A convexity adjusting device for an ostomy skin barrier configured to automatically adjust a convexity of a skin barrier is provided according to various embodiments.

In one aspect, a convex ostomy barrier assembly for attaching an ostomy appliance to a peristomal skin surrounding a stoma may include a skin barrier, an inlet opening defined in the skin barrier for receiving the stoma, and a convexity adjusting device. The convexity adjusting device may be configured to provide a convexity to the skin barrier and to automatically adjust the convexity according to the topography of user's peristomal skin as the convex ostomy barrier assembly is applied to a user. The convexity adjusting device is configured to provide a localized and customized adjustment of at least one characteristic of the convexity.

In an embodiment, the convexity adjusting device may include a plurality of radially extending members configured to define and support the convexity of the ostomy barrier assembly. Each of the plurality of radially extending members may be separated from an adjacent radially extending member by a gap, wherein each of the radially extending members may be configured to be flexed independently according to a force applied to the radially extending member as the skin barrier is pressed against the user's peristomal skin. Each of the radially extending members may be configured to provide an automatic and localized adjustment of at least one of a slope, a depth, a compressibility, and a tension location of the convexity. The convexity adjusting device may include a dome, a base, and a middle portion connecting the dome and the base, wherein the dome may be formed by the plurality of radially extending members.

In an embodiment, the convexity adjusting device may be configured to have a depth of about 3 mm to about 15 mm and a slope of about 15° to about 80°. In another embodiment, the convexity adjusting device may be configured to have a depth of about 7 mm to about 9 mm, a slope of about 55° to about 62°, and may comprise 10-16 radially extending members, wherein each of the radially extending members may be separated by a gap having a width of about 2 mm to about 4 mm.

In an embodiment, the convexity adjusting device may include a convex member and a plurality of beam members. The convex member may include a dome, a base, and a middle portion connecting the dome and the base. The plurality of beam members may be configured to extend radially from an edge of the dome to the base and to provide localized adjustment of a slope of the convexity. Each of the plurality of beam members may be configured to be flexed independently and may comprise at least one hinge configured to facilitate flexing of the beam member. The convexity adjusting device may be initially configured with each of the plurality of beam members extended away from the middle portion, wherein each of the plurality of beam members may be configured to be pressed to flex independently as the skin barrier is pressed against the user's peristomal skin to provide a customized and localized adjustment of the slope.

In an embodiment, the convexity adjusting device comprises 36 beam members. In some embodiments, the middle portion may comprise a plurality of gaps, wherein each of the plurality of gaps may be arranged under each of the plurality of beam members. The plurality of beam members may be integrally formed with the convex member as a single unit or separately formed and attached to the convex member.

In an embodiment, each of the plurality of beam members my include a convex dome portion configured to match a convex body side contour of an adjacent dome area of the convex member.

In an embodiment, the convexity adjusting device may include a mesh portion configured to be flexed and molded automatically to conform to the topography of user's peristomal skin and to provide a self-activating and localized adjustment of a slope, a depth, and/or a tension location of the convexity based on an amount of force applied to the mesh portion by user's peristomal skin. In some embodiments, the mesh portion may be formed from a diamond-pattern mesh. In other embodiments, the mesh portion may be formed from a mesh having various different patterns, for example, circles, ellipses, squares, triangles, and the like. In an embodiment, the convex adjusting device may include an inner rim, an outer rim, and the mesh portion radially extending between the inner rim and the outer rim.

In an embodiment, the convexity adjusting device may include an inner rim, a base, and a middle portion extending therebetween. The middle portion may include a convex dome portion, a concave portion and a plurality of openings and/or grooves. The plurality of openings and/or grooves may include a plurality of long openings and/or grooves radially extending in the convex dome portion and a plurality of short openings and/or grooves in the concave portion. The plurality of openings and/or grooves may include an equal number of the long openings and/or grooves and the short openings and/or grooves, wherein each of the plurality of short openings and/or grooves is aligned with one of the long openings and/or grooves to form a plurality of pairs of long opening/groove and short opening/groove. Each of the pairs of long opening/groove and short opening/groove may extend radially and spaced part from an adjacent pair of long opening/groove and short opening/groove and configured to facilitate bending and flexing of the convex ostomy barrier assembly along the pair of long opening/groove and short opening/groove. In some embodiments, each of the plurality of long openings and/or grooves may be defined by an opening extending entirely through a thickness of the convexity adjusting device, wherein each of the plurality of short openings and/or grooves may be defined by a groove having a depth that is less than a thickness of the convexity adjusting device, such that the groove does not extend through the entire thickness of the convexity adjusting device.

The foregoing general description and the following detailed description are examples only and are not restrictive of the present disclosure. Other aspects, objectives and advantages will become more apparent from the following detailed description when taken in conjunction with the accompanying drawings.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1 is a perspective distal side view of an ostomy barrier appliance including a convexity adjusting device according to an embodiment with a portion removed to illustrate its layered structure.

FIG. 2 is a partial enlarged view of the ostomy barrier appliance of FIG. 1.

FIG. 3A is a perspective body side view of a convexity adjusting device according to an embodiment.

FIG. 3B is a cross sectional view of the convexity adjusting device of FIG. 3A.

FIG. 4 is a perspective distal side view of an ostomy barrier appliance including a convexity adjusting device according to another embodiment with a portion removed to illustrate its layered structure.

FIG. 5 is a partial enlarged view of the ostomy barrier appliance of FIG. 4.

FIG. 6 is a perspective distal view of an ostomy barrier appliance including a convexity adjusting device according to yet another embodiment with a portion removed to illustrate its layered structure.

FIG. 7 is a partial enlarged view of the ostomy barrier appliance of FIG. 6.

FIG. 8 is a perspective distal side view of an ostomy barrier appliance including a convexity adjusting device according to an embodiment with a portion removed to illustrate its layered structure.

FIG. 9 is a partial enlarged view of the ostomy barrier appliance of FIG. 8.

FIG. 10 is an illustration of a depth of a convex skin barrier.

FIG. 11 is an illustration of compressibility of a convex skin barrier.

FIG. 12 is an illustration of flexibility of a convex skin barrier.

FIGS. 13A and 13B are illustrations of tension locations of a convex skin barrier.

FIG. 14 is an illustration of a slope of a convex skin barrier.

FIG. 15A is a perspective body side view of a convexity adjusting device according to an embodiment.

FIG. 15B is a cross sectional view of the convexity adjusting device of FIG. 15A.

FIG. 16A is a perspective distal side view of a convexity adjusting device according to an embodiment.

FIG. 16B is a partial perspective distal side view of the convexity adjusting device of FIG. 16A.

FIG. 17A is a perspective body side view of a convexity adjusting device according to an embodiment.

FIG. 17B is a cross sectional view of the convexity adjusting device of FIG. 17A.

FIG. 18A is a perspective body side view of a convexity adjusting device according to an embodiment.

FIG. 18B is a cross sectional view of the convexity adjusting device of FIG. 18A.

FIG. 19A is a perspective body side view of a convexity adjusting device according to an embodiment.

FIG. 19B is a cross sectional view of the convexity adjusting device of FIG. 19A.

FIG. 20 is a perspective body side view of a convexity adjusting device according to an embodiment.

FIG. 21 is a perspective distal side view of the convexity adjusting device of FIG. 20.

FIG. 22 is a perspective body side view of a convexity adjusting device according to an embodiment.

FIG. 23 is a perspective distal side view of the convexity adjusting device of FIG. 22.

FIG. 24 is a partial cross sectional view of the convexity adjusting device of FIG. 22.

FIG. 25 is a perspective body side view of a convexity adjusting device according to an embodiment.

FIG. 26 is a perspective distal side view of the convexity adjusting device of FIG. 25.

### DETAILED DESCRIPTION

While the present disclosure is susceptible of embodiment in various forms, there is shown in the drawings and will hereinafter be described a presently preferred embodiment with the understanding that the present disclosure is to be considered an exemplification and is not intended to limit the disclosure to the specific embodiment illustrated.

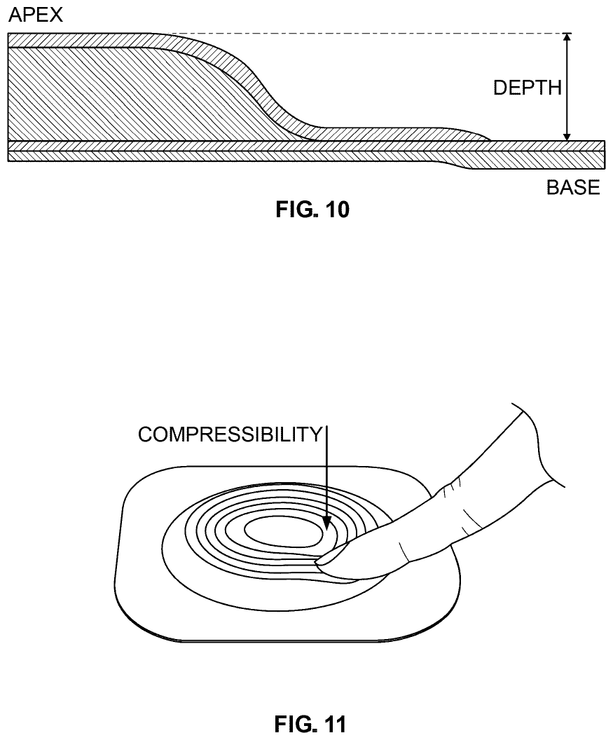

The characteristics of convexity of a skin barrier can include depth, compressibility, flexibility, tension location, and slope. See, McNichol, L., Cobb, T., Depaifve, Y., Quigley, M., Smitka, K., & Gray, M., Characteristics of Convex Skin Barriers and Clinical Application: Results of an International Consensus Panel, J Wound Ostomy Continence Nurs., (2021) 48(6), 524-532, Abstract. The depth of a convex skin barrier is defined as a distance from the apex of the dome to the base of the skin barrier. Id, at pg. 526. The depth D can be measured as a magnitude of the convexity from the base lying on the peristomal skin to the highest point of the skin barrier as shown in FIG. 10. Id. Individual user's peristomal condition, such as depths of creases and folds around the stoma, should be carefully considered when determining a depth of a convex skin barrier to provide an optimal seal around the peristomal skin. Id.

The compressibility of a convex skin barrier is defined as a capacity of the convex dome to be displaced or flattened as illustrated in FIG. 11. Id, at pg. 528. The compressibility may be measured as a force required to displace or flatten the dome portion of a convex skin barrier by a predetermined distance. A relatively easily compressible soft convex barrier may conform better to users with postoperative edema and/or a relatively firm abdominal. Id. A relatively less compressible firm convex barrier may apply more pressure on the peristomal skin to provide support needed for users with a relatively soft abdominal tone and/or creases around the stoma. Id.

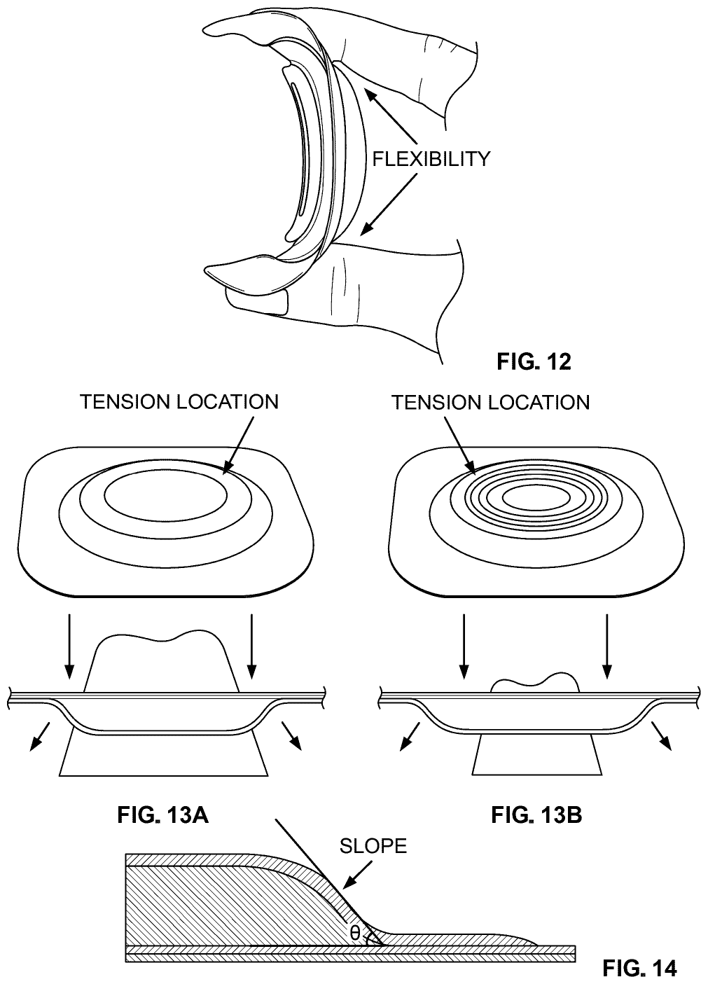

The flexibility of a convex skin barrier is defined as how easily the convex skin barrier can bend, as illustrated in FIG. 12. Id, at pg. 529. The flexibility is an important characteristic to consider when a skin barrier needs to bend to conform to abdominal contours. Id. A relatively more flexible convex skin barrier may work well for users with multiple creases around stoma due to loose skin. Id.

The tension location of a convex skin barrier is defined as the position in which a convex dome exerts downward and outward forces on the peristomal topography, as illustrated in FIGS. 13A and 13B. Id, at pg. 530. A convex skin barrier configured to apply a tension close to a stoma may provide a consistent and reliable seal around the stoma that is flush to the skin or retraced below the skin. Id. For users with creases and folds around the stoma, a convex barrier skin barrier configured to apply a tension away from the stoma may help flatten the peristomal skin to provide a good seal. Id.

The slope of a convex skin barrier is defined as an angle from the base of the dome to a periphery of the apex of the dome, as illustrated in FIG. 14. Id, at pg. 53. Creases and folds around the stoma can compromise a seal between a skin barrier and the skin. Adjusting the slope of a convex skin barrier according to user's peristomal topography can improve the seal. For example, a convex skin barrier with a relatively small slope and wider plateau may help flatten the peristomal skin creases and folds to achieve a good seal. Id.

Customizing and adjusting the depth, compressibility, flexibility, tension location, and/or slope of a convex skin barrier according to user's peristomal topography can provide an optimal seal around the stoma. The present disclosure provides an ostomy barrier appliance including a convexity adjusting device configured to automatically adjust one or more of the convexity characteristics of a skin barrier according to various embodiments. The convexity adjusting device may be configured as a self-activating device, which may be automatically adjusted and formed according to user's peristomal topography as the ostomy barrier appliance is applied to the user.

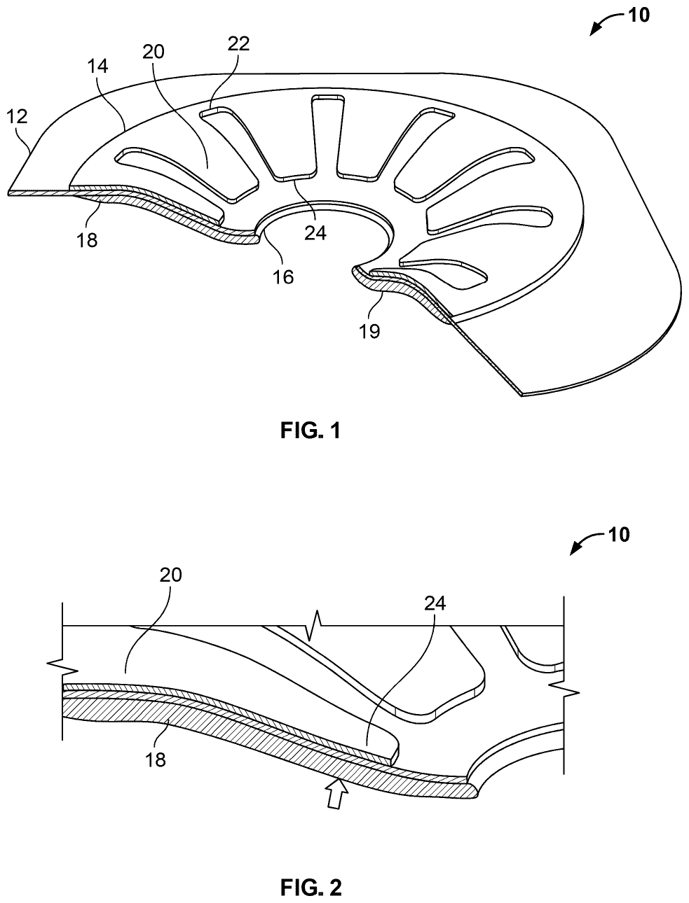

Referring now to the figures, FIGS. 1-2 show an ostomy barrier appliance 10 according to an embodiment. FIG. 1 is a perspective view of the ostomy barrier appliance 10 with a portion removed and viewed from a pouch-facing side (also referred to herein as a distal side) to illustrate a layered construction of the ostomy barrier appliance 10. The ostomy barrier appliance 10 may generally include a tape 12, a skin barrier 18, a convexity adjusting device 14, and an inlet opening 16 for receiving a stoma. The convexity adjusting device 14 may include radially extending members 20 separated by gaps 22, which may be configured to automatically adjust the convexity of the ostomy barrier appliance 10 without user interaction and provide an adaptable seal. The radially extending members 20 include ends 24 near inlet opening 16. The convexity adjusting device 14 may be attached to a distal side of the skin barrier 18 or provided as a convex insert that may be inserted into the ostomy barrier appliance 10.

The tape 12 may include a first adhesive layer and a first backing layer. The first adhesive layer of the tape 12 may be formed from a suitable medical adhesive, such as an acrylic adhesive. The first backing layer may be formed from a suitable material, such as a nonwoven material or a thin polymeric film. In another embodiment, the tape 12 may comprise a hydrocolloid adhesive and a film backing layer.

In some embodiments, the skin barrier 18 may include a second backing layer laminated on the pouch-side surface of the skin barrier 18. The second backing layer may be formed from a suitable heat sealable polymeric material, such that the backing layer may be heat sealed to the tape 12. The skin barrier 18 may be formed from a suitable skin-contact adhesive, such as hydrocolloid adhesives.

In an embodiment, the convexity adjusting device 14 may include a plurality of radially extending members 20, which are also referred to herein as petals. The plurality of petals 20 may be configured to define and support a convexity of the skin barrier 18. Each of the plurality of petals 20 may be separated from an adjacent petal 20 by a gap 22, such that each petal 20 may be adjusted relatively independent from each other to allow localized adjustment of at least one characteristic of the convexity of the ostomy barrier appliance 10 to better accommodate the area around the stoma according to the peristomal topography. For example, the plurality of petals 20 may be configured to provide a localized and customized adjustment of a slope, depth, and/or tension location of the convex skin barrier 18.

In an embodiment, the ostomy barrier appliance 10 may be configured as a convex ostomy barrier including a dome 19 supported by the plurality of petals 20, wherein each of the plurality of petals 20 may be formed to provide a uniform initial convexity depth and slope around the dome 19 as shown in FIG. 1. In use, each of the plurality of petals 20 may be independently adjusted and shaped to conform to user's peristomal topography as the ostomy barrier appliance 10 is attached to the user. The convexity adjusting device 14 may be configured such that each of the plurality of petals 20 may be flexed, compressed and/or formed independently as the skin barrier 18 is pressed against the peristomal skin to provide a customized and localized convexity of the ostomy barrier appliance 10. FIG. 2 illustrates one of the plurality of petals 20 being pressed and flattened by a force applied at a direction shown by the arrow, for example, as the petal 20 is pressed against a protruding portion of the peristomal skin (not shown). Further, a user can adjust the orientation of the ostomy barrier appliance 10, such that one or more of the gaps 22 may be arranged across an axis where more flexibility is desired.

The convexity adjusting device 14 may be formed from a suitable material, such as polymeric materials, rubber, silicone, or metallic materials. For example, the convexity adjusting device 14 may be formed from a heat sealable thermoplastic material, such as ethylene vinyl acetate (EVA), thermoplastic elastomer, or thermoplastic urethane. In another example, the convexity adjusting device 14 may be formed from a foam or silicone. The convexity adjusting device 14 may be formed via thermoforming or other known methods.

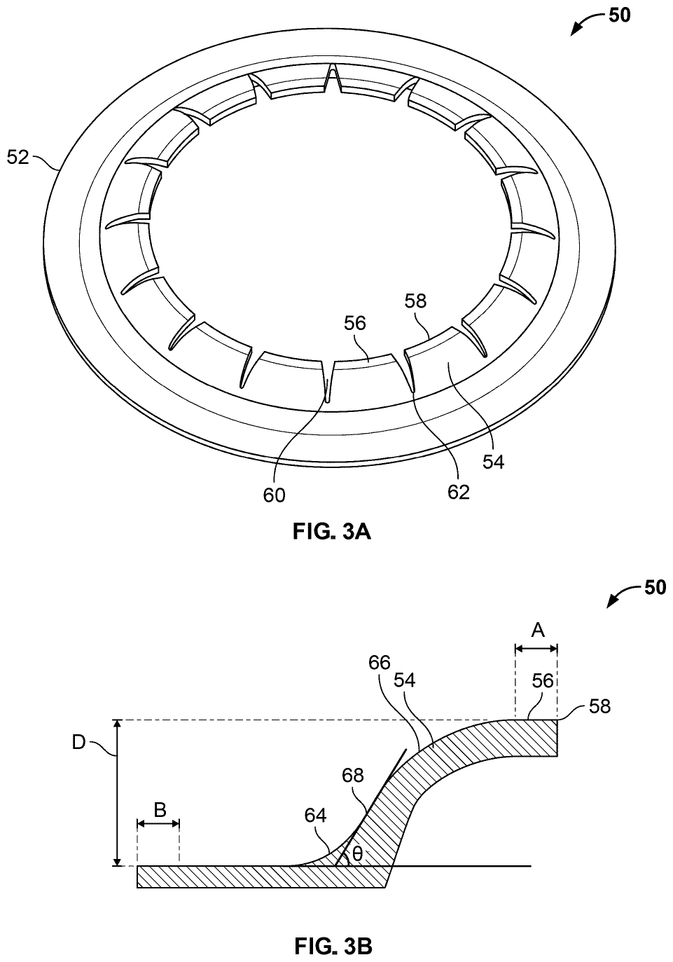

FIGS. 3A and 3B show a convexity adjusting device 50 according to an embodiment. FIG. 3A is a perspective body side view of the convexity adjusting device 50, and FIG. 3B is a cross-sectional view of the convexity adjusting device 50. The convexity adjusting device 50 may be similarly constructed to the convexity adjusting device 14 and may be attached to an ostomy barrier appliance 10 that generally includes a tape 12, a skin barrier 18 and an inlet opening 16 for receiving a stoma. The convexity adjusting device 50 may include an outer portion 52 (also referred to herein as a base) and a dome 54 that extends from the outer portion 52 and protrudes toward a body side. The dome 54 may be defined by a plurality of radially extending members 56, wherein each of the plurality of radially extending members 56 may be separated by a gap 60 (also referred to herein as a slit). Each of the radially extending members 56 may include a peripheral end 58 proximate the inlet opening 16. In this embodiment, each of the gaps 60 may be defined by a generally v-shape opening, wherein a width of the gap 60 may be maximum at a first end proximate the peripheral end 58 and may decrease therefrom towards an end 62 of the gap 60 as best shown in FIG. 3A.

As best shown in FIG. 3B, the convex adjusting device 50 may include a concave body side portion 64 and a convex body side portion 66, wherein the dome 54 is defined in the convex body side portion 66. The convexity adjusting device 50 may be configured to have a convexity depth D of about 3 mm to about 15 mm and a convexity slope θ of about 15° to about 80°. The convexity slope θ is a maximum slope measured on the body side surface of the convex adjusting device 50. In the embodiment of FIG. 3B, the convexity slope θ is measured at a transition point 68 between the concave body side portion 64 and the convex body side portion 66. In an embodiment, the convexity adjusting device 50 may be configured to have a convexity depth D of about 7 mm (FIG. 3B), including 16 radially extending members 56. The convexity adjusting device 50 may be attached to a distal side of the skin barrier 18 and/or the tape 12. For example, the convexity adjusting device 50 may be heat sealed to the skin barrier 18 and/or the tape 12 along an inner diameter sealing area A and an outer diameter sealing area B (FIG. 3B).

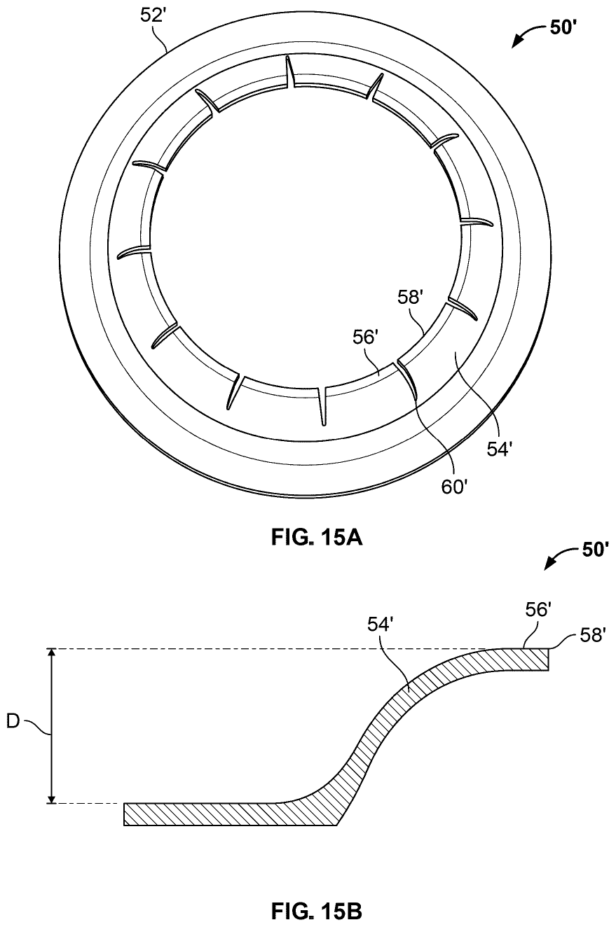

In another embodiment, the convexity adjusting device 50′ may be configured to have a convexity depth D of about 7 mm including 12 radially extending members 56′ as shown in FIGS. 15A and 15B.

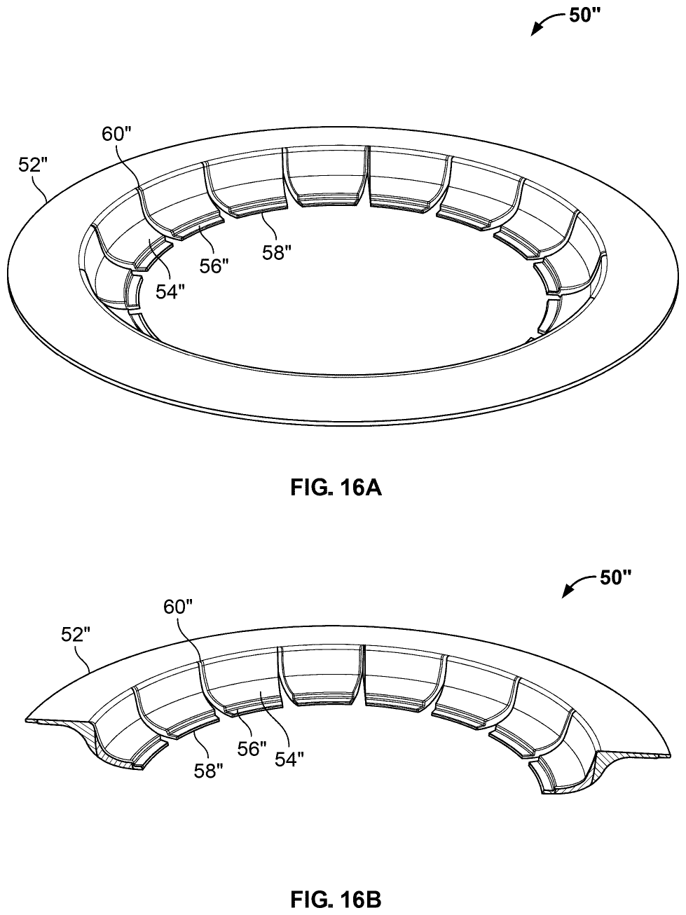

In yet another embodiment, the convexity adjusting device 50″ may be configured to have a convexity depth of about 9 mm including 16 radially extending members 56″ as shown in FIGS. 16A and 16B. The convexity adjusting device 50″ may be configured to have a convexity slope of about 62°.

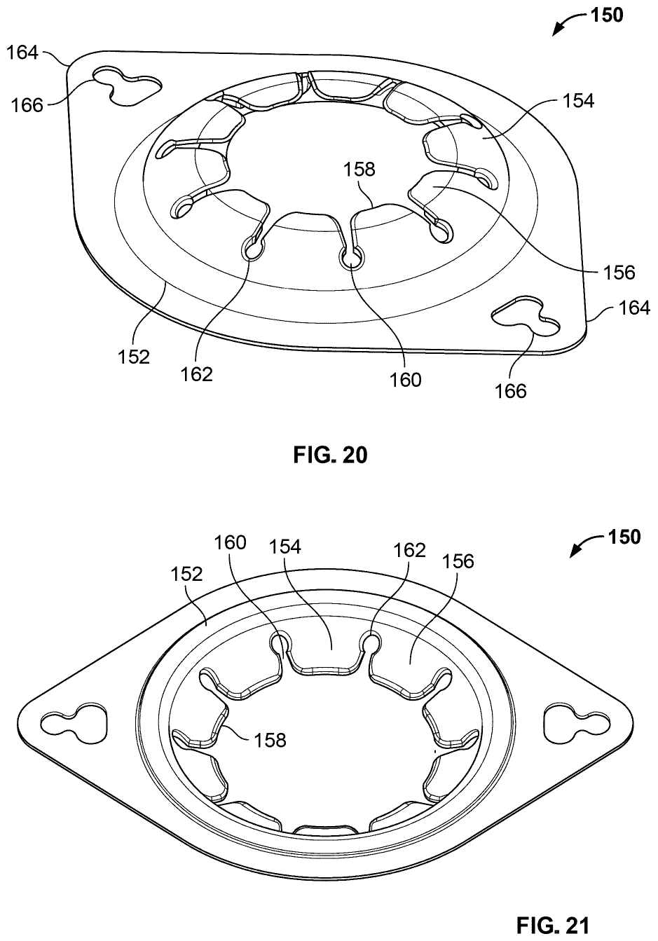

FIGS. 20 and 21 show a convexity adjusting device 150 according to an embodiment. FIG. 20 is a perspective body side view of the convexity adjusting device 150, and FIG. 21 is a perspective distal side view of the convexity adjusting device 150. The convexity adjusting device 150 may be similarly constructed to the convexity adjusting device 14, 50 and may be attached to an ostomy barrier appliance 10 that generally includes a tape 12, a skin barrier 18 and an inlet opening 16 for receiving a stoma. The convexity adjusting device 150 may include an outer portion 152 (also referred to herein as a base) and a dome 154 that extends from the base 152 and protrudes toward a body side. The dome 154 may be defined by a plurality of radially extending members 156, wherein each of the plurality of radially extending members 156 may be separated by a gap 160. Each of the radially extending members 156 may include a peripheral end 158 proximate the inlet opening 16.

In this embodiment, each of the gaps 160 may include a generally oval shape or rounded end 162, wherein a width of the gap 160 may decrease from a first end proximate the peripheral end 158 towards the rounded end 162 and increase in the rounded end 162 as shown in FIGS. 20 and 21. The increased width of the gap 160 in the rounded end 162 may improve compressibility and/or flexibility of each of the radially extending member 156 when compared to the convex adjusting device 50 including the generally v-shaped gaps 60. The base 152 may include extended side wings 164 and openings 166 defined therein for engaging with ostomy belt coupling members (not shown).

The convexity adjusting device 50, 50′, 50″, 150 may be configured to operate similar to the convexity adjusting device 14, wherein each of the radially extending members 56, 56′, 56″, 156 may be independently adjusted and shaped to conform to user's peristomal topography as the ostomy barrier appliance 10 is applied to the user.

The convexity adjusting device 50, 50′, 50″, 150 may include a base 52, 52′, 52″, 152 and a dome 54, 54′, 54″, 154. The dome 54, 54′, 54″, 154 may be defined by a plurality of radially extending members 56, 56′, 56″, 156 wherein each of the plurality of radially extending members 56, 56′, 56″, 156 may be separated by a gap 60, 60′, 60″, 160. Each of the radially extending members 56, 56′, 56″, 156 may include a peripheral end 58, 58′, 58″, 158 proximate the inlet opening 16.

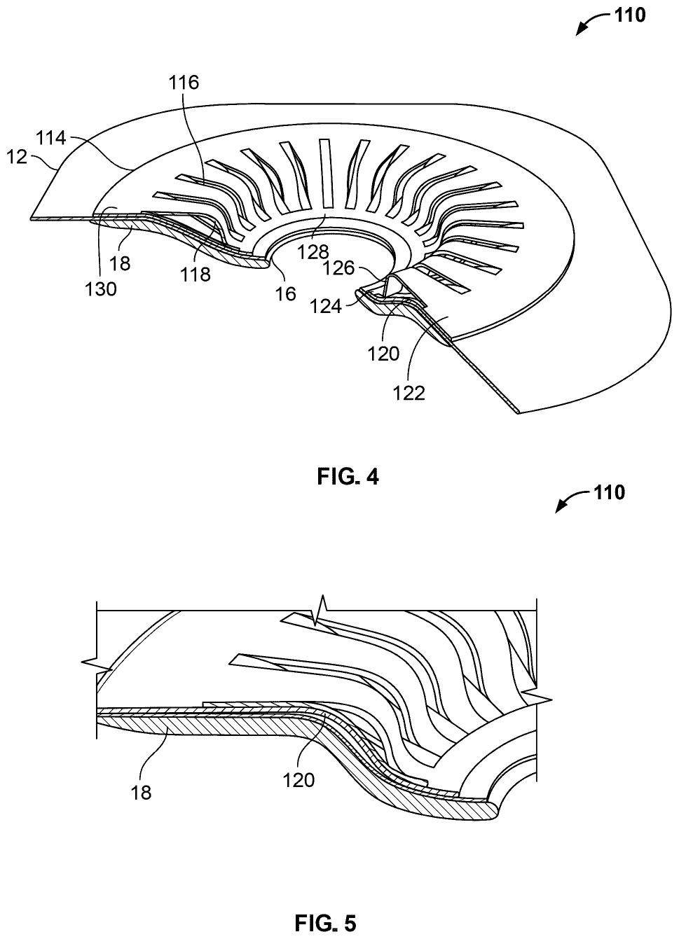

FIGS. 4 and 5 show an ostomy barrier appliance 110 according to an embodiment. The ostomy barrier appliance 110, may be configured similar to ostomy barrier appliance 10 of FIG. 1, and may generally include a tape 12, a skin barrier 18, a convexity adjusting device 114, and an inlet opening 16 for receiving a stoma. The convexity adjusting device 114 may include a convex member 118 and a plurality of beam members 120. The convex member 118 may include a base 122, a dome 124, and a middle portion 126. The convex member 118 may be attached to a distal side of the skin barrier 18 and/or tape 12 along an inner peripheral portion 128 and an outer peripheral portion 130. The plurality of beam members 120 may be configured to extend radially between an edge of the dome 124 and the base 122 and configured for localized adjustment of the convexity of the ostomy barrier appliance 10, for example, a convexity slope. The plurality of beam members 120 may be integrally formed with the convex member 118 or separately formed and attached to the convex member 118.

In the embodiment of FIGS. 4 and 5, the plurality of beam members 120 may be integrally formed with the convex member 118 as a single unit. The plurality of beam members 120 may be defined between a plurality of cuts formed in the middle portion 126. Each of the plurality of beam members 120 may be pushed out or extended away from the middle portion 126 to decrease the convexity slope at the corresponding location as shown in FIG. 4. In such an embodiment, a plurality of gaps 116 may be formed in the middle portion 126 under the plurality of the beam members 120 as shown in FIG. 4.

The convexity adjusting device 114 may be configured such that each of the plurality of beam members 120 may be flexed independently. In an embodiment, the convexity adjusting device 114 may be initially configured with each of the plurality of beam members 120 extended away from the middle portion 126 as shown in FIG. 4. In use, each of the plurality of beam members 120 may be independently pressed to flex as the skin barrier 18 is pressed against the peristomal skin and provide a customized and localized adjustment of the convexity slope. FIG. 5 illustrates one of the plurality of beam members 120 flexed toward a distal side as the beam member 120 is pressed against a protruding portion of the peristomal skin (not shown), thereby providing a localized and customized increase of the convexity slope according to the topography of the peristomal skin.

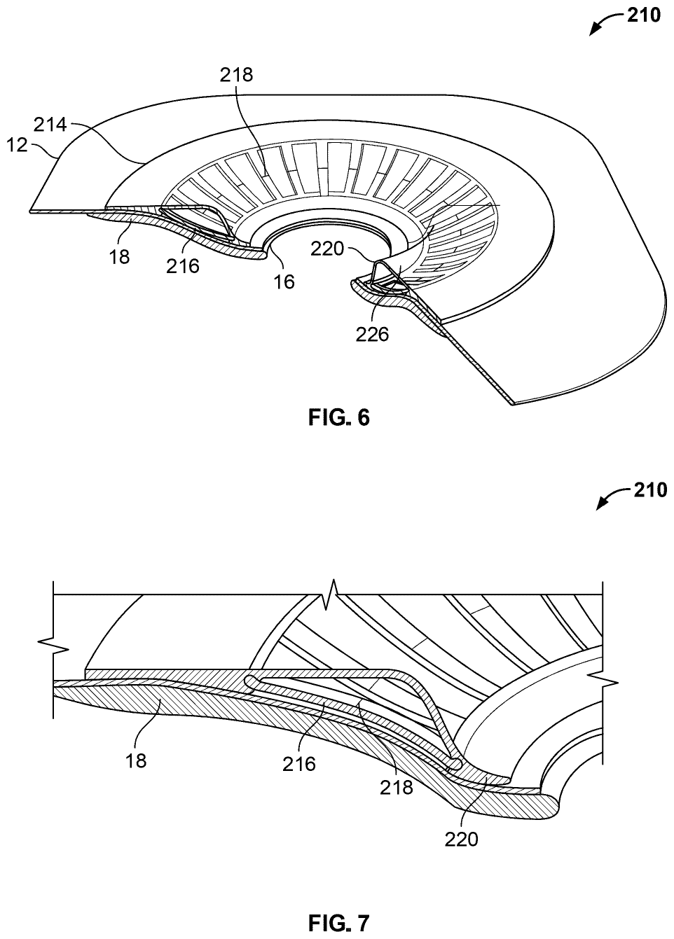

FIGS. 6 and 7 show an ostomy barrier appliance 210 according to another embodiment. The ostomy barrier appliance 210 may be configured similar to the ostomy barrier appliance 110 of FIG. 4, and may generally include a tape 12, a skin barrier 18, a convexity adjusting device 214, and an inlet opening 16 for receiving a stoma. The convexity adjusting device 214 may be configured similar to the convexity adjusting device 114 and may include a convex member 220 and a plurality of beam members 216, except the convex member 220 does not include gaps in the middle portion 226. In this embodiment, the plurality of beam members 216 may be formed separately and attached to the convex member 220 as best shown in FIG. 7. Each of the beam members 216 may include a hinge 218 configured to facilitate flexing of the beam members 216 as the ostomy barrier appliance 210 is applied to a user. The hinge 218 may be formed on a distal side and/or a body side proximate a center of the beam members 216. In an embodiment, the hinge 218 may be formed on a body side of the beam member 216 and another hinge may be formed on a distal side opposite the hinge 218.

In an embodiment, the convex member 220 does not include gaps in the middle portion 226 and the convex member 220 may act as a cover that prevents fluid or other matters from entering the convexity adjusting device 214 and making contact with the beam members 216. For example, when the convexity adjusting device 214 is secured around a stoma, no fluid or other matters may enter the convexity adjusting device 214 when backflow occurs in the ostomy bag because of the ostomy bag being overfilled or lifted up by a user.

In an embodiment, the convexity adjusting device 214 may be configured to automatically adjust the convexity slope of the skin barrier 18 according to the peristomal topography when the ostomy barrier appliance 210 is secured around a stoma. The convexity adjusting device 214 may be configured to automatically recover its initial configuration when removed from around the stoma.

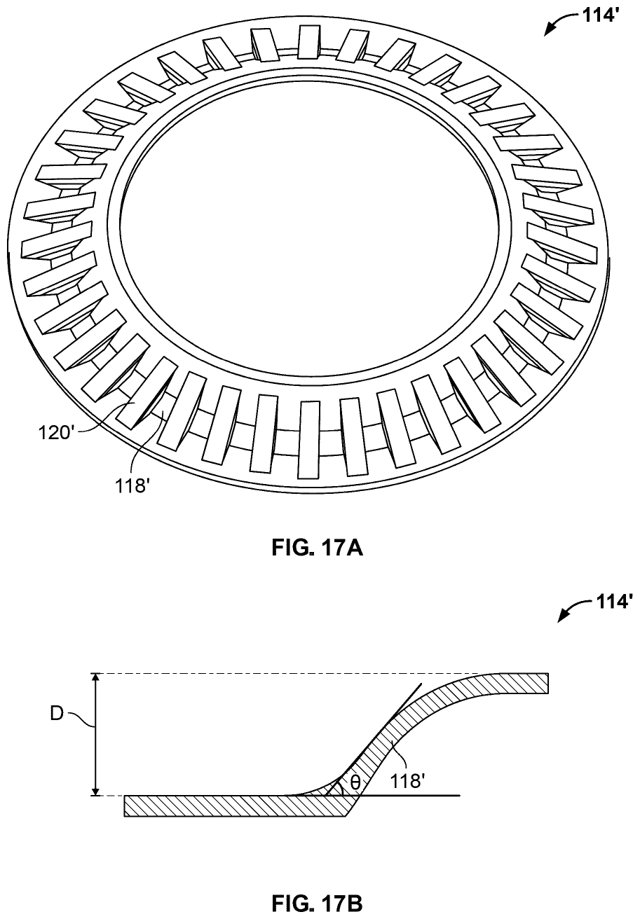

FIGS. 17A and 17B show a convexity adjusting device 114′ according to an embodiment. FIG. 17A is a perspective body side view of the convexity adjusting device 114′, and FIG. 17B is a cross-sectional view of the convexity adjusting device 114′. The convexity adjusting device 114′ may be similarly configured to the convexity adjusting device 114 and may include a convex member 118′ and a plurality of beam members 120′. The convex adjusting device 114′ may be configured to provide a convexity having a depth D of about 3 mm to about 15 mm, a convexity slope θ of about 15° to about 80°, and a tension location at about 1 mm to about 50 mm radially spaced from the stoma. In an embodiment, the convex member 118′ may be configured to have a depth D of about 6 mm (FIG. 17B). The plurality of beam members 120′ may include 36 beam members configured to support the skin barrier 18 without creating ripple look or feel in the ostomy barrier appliance, while enabling localized slope adjustment.

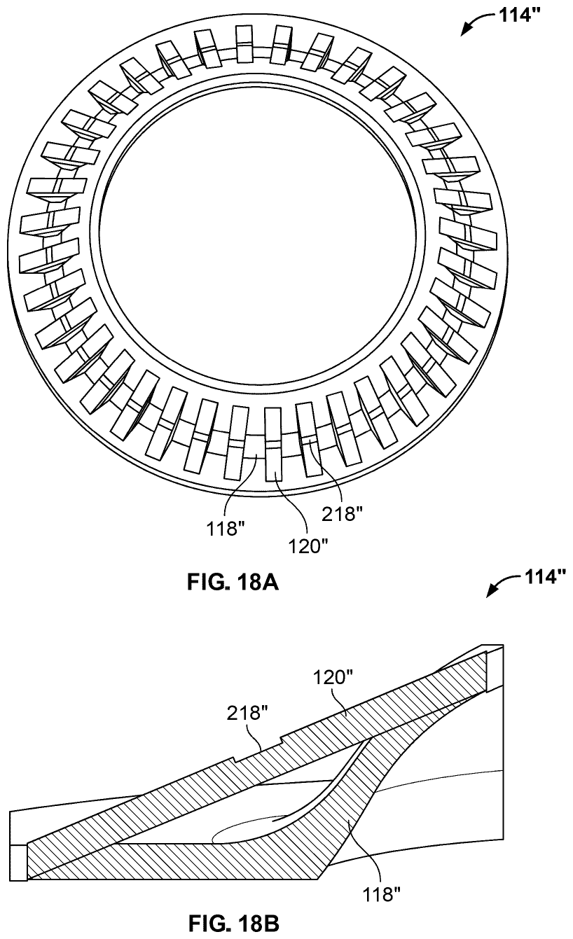

FIGS. 18A and 18B show a convexity adjusting device 114″ according to an embodiment. FIG. 18A is a perspective body side view of the convexity adjusting device 114″, and FIG. 18B is a cross-sectional view of the convexity adjusting device 114″. The convexity adjusting device 114″ may be similarly configured to the convexity adjusting device 114 and may include a convex member 118″ and a plurality of beam members 120″. In this embodiment, each of the plurality of beam members 120″ may include a hinged zone 218″. The hinged zone 218″ may be formed proximate a center of each of the beam members 120″ on a body side as shown in FIGS. 18A and 18B. In other embodiments, the hinged zone 218″ may be formed on a distal side of each of the beam members 120″ or formed on both distal and body sides of the beam members 120″.

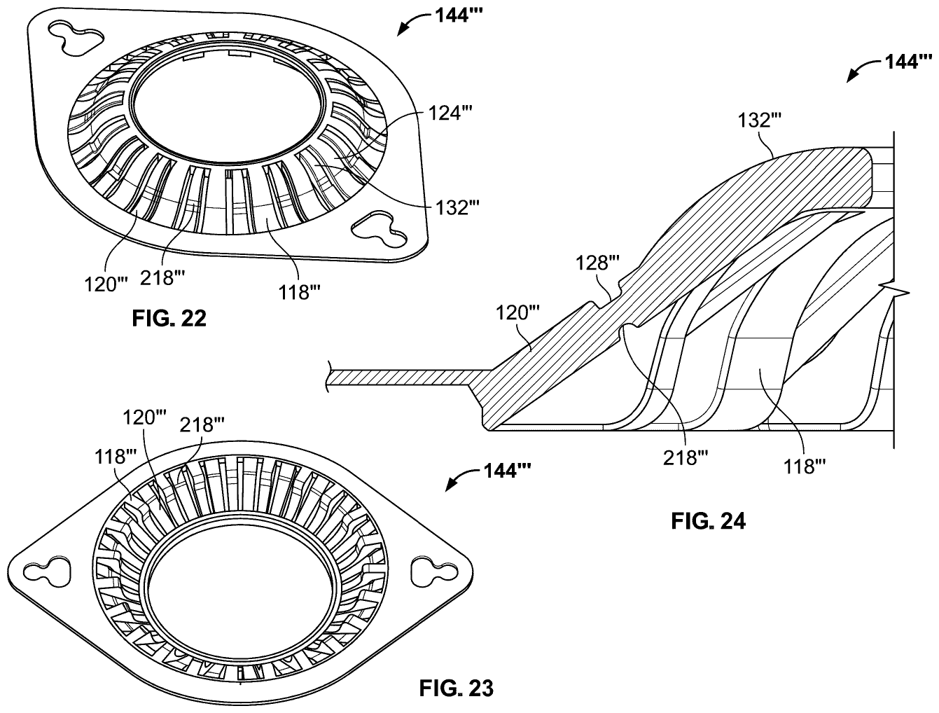

FIGS. 22-24 show a convexity adjusting device 114′″ according to an embodiment. FIG. 22 is a perspective body side view of the convexity adjusting device 114′″, FIG. 23 is a perspective distal side view of the convexity adjusting device 114′″, and FIG. 24 is a partial cross-sectional view of the convexity adjusting device 114′″. The convexity adjusting device 114′″ may be similarly configured to the convexity adjusting device 114″ and may include a convex member 118′″ and a plurality of beam members 120′″. Each of the plurality of beam members 120′″ may include a hinged zone 218′″ configured to facilitate flexing of the beam member 120′″ as the ostomy barrier appliance is applied to a user. The hinged zone 218′″ may be formed on body side and distal side of each of the beam members 120′″ as best shown in FIG. 24. In other embodiments, the hinged zone 218′″ may be formed only on the distal side of each of the beam members 120″ or formed only on the body sides of the beam members 120′″. In this embodiment, each of the beam members 120′″ includes a convex dome portion 132′″ configured to generally match a convex body side contour of adjacent dome 124′″ areas of the convex member 118″.

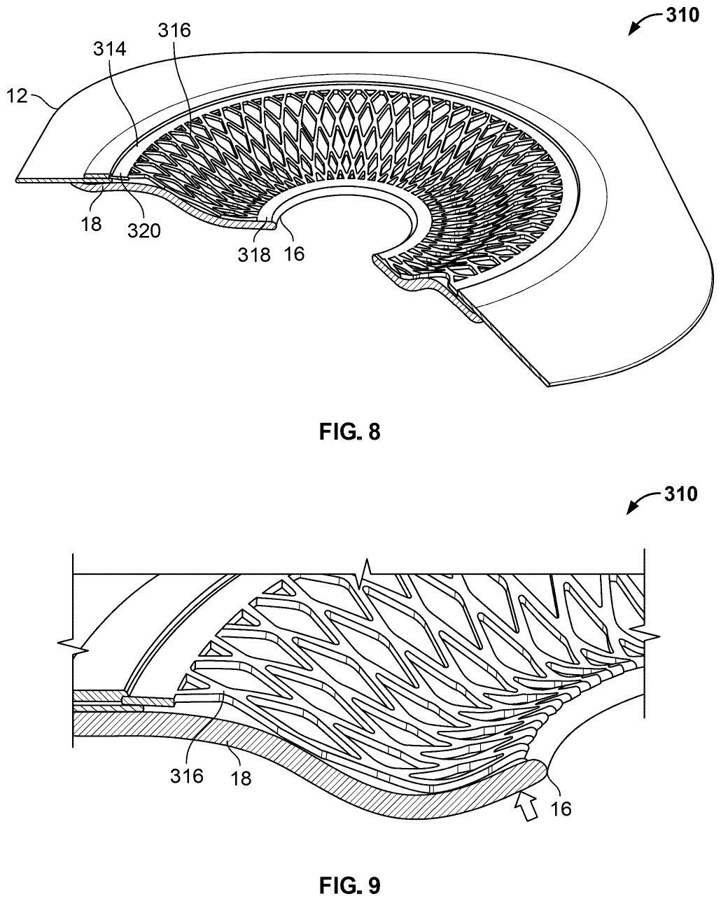

FIGS. 8 and 9 show an ostomy barrier appliance 310 according to an embodiment. The ostomy barrier appliance 310 may be configured similar to the ostomy barrier appliance 10 of FIG. 1, and may generally include a tape 12, a skin barrier 18, a convexity adjusting device 314, and an inlet opening 16 for receiving a stoma. The convexity adjusting device 314 may include an inner rim 318, an outer rim 320, and a mesh portion 316 radially extending therebetween. The mesh portion 316 may include a mesh pattern configured to allow the convexity adjusting device 314 to be flexed and molded according to the topography of user's peristomal skin as the ostomy barrier appliance 310 is applied to the user. The convexity adjusting device 314 may be configured to achieve localized and customized adjustment of a slope, depth, and/or tension location of the skin barrier 18 based on how force is applied to the convexity adjusting device 314 as the ostomy barrier appliance 310 is applied to the user. In an embodiment, the convexity adjusting device 314 may be configured to provide a convexity having a depth D of about 3 mm to about 15 mm, a convexity slope of about 15° to about 80°, and a tension location at about 1 mm to about 50 mm radially spaced from the stoma.

In an embodiment, the mesh portion 316 may be formed from a diamond-pattern mesh configured to automatically adjust the convexity of the skin barrier 18 according to the user's peristomal topography as the ostomy barrier appliance 310 is secured around the stoma. The convexity adjusting device 314 may be configured to provide a localized and customized change in slope, softness and/or depth of the skin barrier 18 based on a force applied by the topograph of user's skin. FIG. 9 shows the ostomy barrier appliance 310 being pressed proximate an inner periphery in response to a force (shown with an arrow) applied by user's peristomal skin (not shown) as the ostomy barrier appliance 310 is attached to the user. In an embodiment, the convexity adjusting device 314 may not include the inner rim 318 as shown in FIG. 9. In such an embodiment, an inner periphery of the convexity adjusting device 314 may be defined by an inner periphery of the mesh portion 316 to provide more flexibility to seal around a stoma.

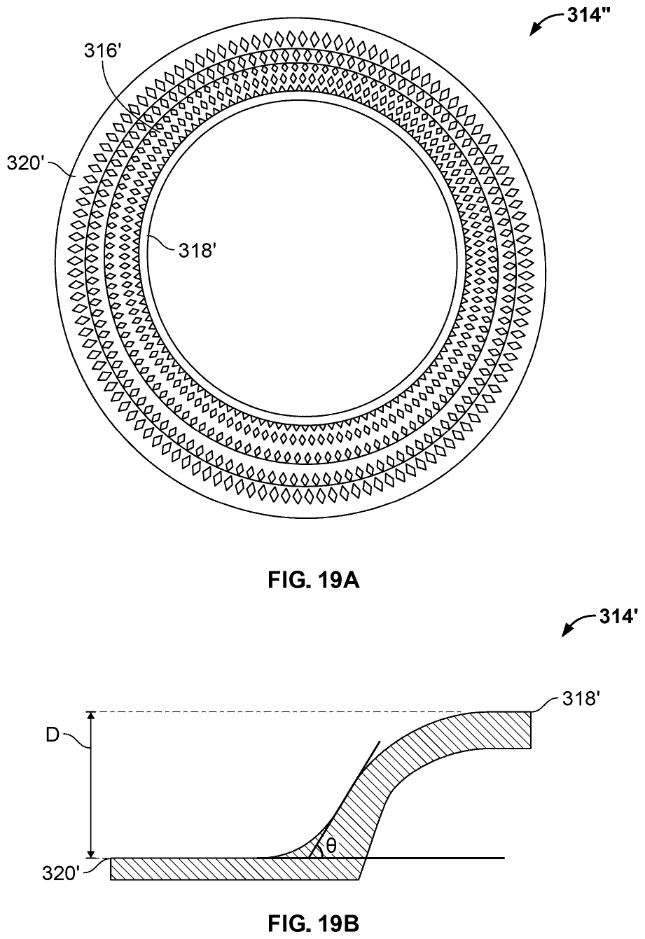

FIGS. 19A and 19B show a convexity adjusting device 314′ according to an embodiment. FIG. 19A is a body side view of the convexity adjusting device 314′, and FIG. 19B is a cross-sectional view of the convexity adjusting device 314′. The convexity adjusting device 314′ may be similarly configured to the convexity adjusting device 314 and may include mesh portion 316′, inner rim 318′, and outer rim 320′. The convexity adjusting device 314′ may be configured to provide a convexity having a depth D of about 3 mm to about 15 mm, a convexity slope θ of about 15° to about 80°, and a tension location at about 1 mm to about 50 mm radially spaced from the stoma. In an embodiment, the convex member 314′ may be configured to have a depth D of about 7 mm (FIG. 19B).

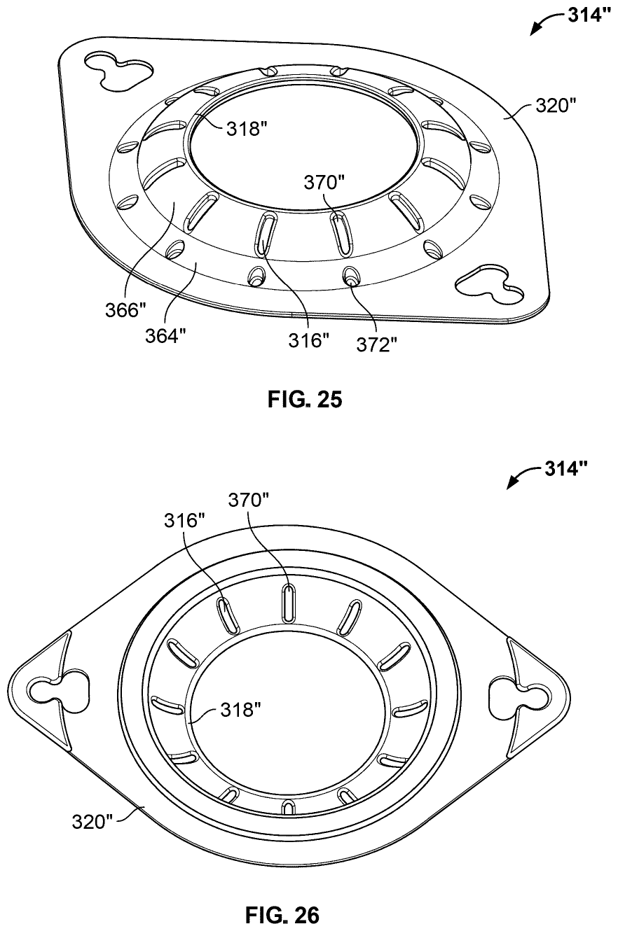

FIGS. 25 and 26 show a convexity adjusting device 314″ according to an embodiment. FIG. 25 is a body side view of the convexity adjusting device 314″, and FIG. 26 is a distal side view of the convexity adjusting device 314″. The convexity adjusting device 314″ may be similarly configured to the convexity adjusting device 314′ and may include an inner rim 318″ and outer rim 320″ (also referred to herein as a base). In this embodiment, the convexity adjusting device 314″ may include a middle portion comprising a plurality of openings and/or grooves 316″ instead of the mesh portion 316′. The plurality of openings and/or grooves 316″ may include a plurality of long openings and/or grooves 370″ radially extending in a convex dome portion 366″ and a plurality of short openings and/or grooves 372″ in a concave middle portion 364″ of the convexity adjusting device 314″.

In an embodiment, the plurality of openings and/or grooves 316″ may include the same number of the long openings and/or grooves 370″ and the short openings and/or grooves 372″, wherein each of the plurality of short openings and/or grooves 372″ may be aligned with a corresponding long opening/groove 370″ as shown in FIG. 25. In such an embodiment, each pair of the long opening/groove 370″ and short opening/groove 372″, which extend radially and spaced part from adjacent pairs, may be configured to facilitate bending and flexing of an ostomy barrier appliance including the convexity adjusting device 314″ along the pairs of long opening/groove 370″ and short opening/groove 372″. In the embodiment of FIGS. 25 and 26, each of the plurality of long openings/grooves 370″ may be an opening formed entirely through the thickness of the convexity adjusting device 314″, and each of the plurality of short openings/grooves 372″ may be formed as a groove having a depth that is less than the thickness of the convexity adjusting device 314″, such that the groove 372″ does not extend through the entire thickness of the convexity adjusting device 314″. In some embodiments, the plurality of long openings and/or grooves 370″ and the plurality of short openings and/or grooves may include only openings, only grooves, or a mixture of openings and grooves.

All patents referred to herein, are hereby incorporated herein in their entirety, by reference, whether or not specifically indicated as such within the text of this disclosure.

In the present disclosure, the words “a” or “an” are to be taken to include both the singular and the plural. Conversely, any reference to plural items shall, where appropriate, include the singular.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

What is claimed is:

1. A convex ostomy barrier assembly for attaching an ostomy appliance to a peristomal skin surrounding a stoma, comprising:
a skin barrier comprising an adhesive;
an inlet opening defined in the skin barrier for receiving the stoma; and
a convexity adjusting device, wherein the convexity adjusting device is configured to provide a convexity to the skin barrier and to adjust the convexity according to a topography of user's peristomal skin as the convex ostomy barrier assembly is applied to a user, and wherein the convexity adjusting device is configured to provide a localized and customized adjustment of at least one characteristic of the convexity.

2. The convex ostomy barrier assembly of claim 1, wherein the convexity adjusting device comprises a plurality of radially extending members configured to define and support the convexity, wherein each of the plurality of radially extending members is separated from an adjacent radially extending member by a gap, wherein each of the radially extending members is configured to be flexed independently according a force applied to each of the radially extending member as the skin barrier is pressed against the user's peristomal skin, and wherein each of the radially extending members is configured to provide an automatic and localized adjustment of at least one of a slope, a depth, and/or a tension location of the convexity.

3. The convex ostomy barrier assembly of claim 2, wherein the convexity adjusting device comprises a dome, a base, and a middle portion connecting the dome and the base, wherein the dome is formed by the plurality of radially extending members.

4. The convex ostomy barrier assembly of claim 3, wherein the convexity adjusting device has a depth of about 7 mm, a slope of about 55°, and comprises 12 radially extending members.

5. The convex ostomy barrier assembly of claim 3, wherein the convexity adjusting device has a depth of about 7 mm to about 9 mm, a slope of about 55° to about 62°, and comprises 16 radially extending members.

6. The convex ostomy barrier assembly of claim 1, wherein the convexity adjusting device comprises a convex member and a plurality of beam members, wherein the convex member includes a dome, a base, and a middle portion connecting the dome and the base, wherein each of the plurality of beam members extends radially and configured to be flexed independently to provide localized adjustment of a slope of the convexity.

7. The convex ostomy barrier assembly of claim 6, wherein each of the plurality of beam members comprises at least one hinge configured to facilitate flexing of the beam member.

8. The convex ostomy barrier assembly of claim 6, wherein the convexity adjusting device is initially configured with each of the plurality of beam members extended away from the middle portion, and wherein each of the plurality of beam members is configured to be pressed to flex independently as the skin barrier is pressed against the user's peristomal skin to provide a customized and localized adjustment of the slope.

9. The convex ostomy barrier assembly of claim 6, wherein the convexity adjusting device comprises 36 beam members.

10. The convex ostomy barrier assembly of claim 6, wherein the middle portion comprises a plurality of gaps, wherein each of the plurality of gaps is arranged under each of the plurality of beam members.

11. The convex ostomy barrier assembly of claim 6, wherein the middle portion and the plurality of beam members are integrally formed as a single unit.

12. The convex ostomy barrier assembly of claim 6, wherein the plurality of beam members are separately formed and attached to the middle portion.

13. The convex ostomy barrier assembly of claim 6, wherein each of the plurality of beam members includes a convex dome portion configured to match a convex body side contour of an adjacent dome area of the convex member.

14. The convex ostomy barrier assembly of claim 1, wherein the convexity adjusting device comprises a mesh portion configured to be flexed and molded automatically to conform to the topography of user's peristomal skin and to provide a self-activating and localized adjustment of a slope, a depth, and/or a tension location of the convexity based on an amount of force applied to the mesh portion by user's peristomal skin.

15. The convex ostomy barrier assembly of claim 14, wherein the mesh portion is formed from a diamond-pattern mesh.

16. The convex ostomy barrier assembly of claim 14, wherein the mesh portion comprises mesh openings, wherein the mesh openings near the inlet opening are smaller than the mesh openings away from the inlet opening.

17. The convex ostomy barrier assembly of claim 14, wherein the convexity adjusting device has a depth of about 7 mm and comprises 120 mesh openings.

18. The convex ostomy barrier assembly of claim 14, wherein the convex adjusting device includes an inner rim, an outer rim, and the mesh portion radially extending between the inner rim and the outer rim.

19. The convex ostomy barrier assembly of claim 1, wherein the convexity adjusting device comprises an inner rim, a base, and a middle portion extending between the inner rim and the base, wherein the middle portion includes a convex dome portion, a concave portion and a plurality of openings and/or grooves, wherein the plurality of openings and/or grooves include a plurality of long openings and/or grooves radially extending in the convex dome portion and a plurality of short openings and/or grooves in the concave portion.

20. The convex ostomy barrier assembly of claim 19, wherein the plurality of openings and/or grooves includes an equal number of the long openings and/or grooves and the short openings and/or grooves, wherein each of the plurality of short openings and/or grooves is aligned with one of the long openings and/or grooves to form a plurality of pairs of long opening/groove and short opening/groove, wherein each of the pairs of long opening/groove and short opening/groove extends radially and spaced part from an adjacent pair of long opening/groove and short opening/groove and configured to facilitate bending and flexing of the convex ostomy barrier assembly along the pair of long opening/groove and short opening/groove.

21. The convex ostomy barrier assembly of claim 19, wherein each of the plurality of long openings and/or grooves is an opening extending entirely through a thickness of the convexity adjusting device, and each of the plurality of short openings and/or grooves is a groove having a depth that is less than a thickness of the convexity adjusting device, such that the groove does not extend through the entire thickness of the convexity adjusting device.

22. The convex ostomy barrier assembly of claim 1, wherein the convexity adjusting device is configured to provide the convexity having a depth of about 3 mm to about 15 mm, a slope of about 15° to about 80°, and a tension location at about 1 mm to about 50 mm radially spaced from the stoma.
